(19) United States
(12) Patent Application Publication
(10) Pub. No.: US 2013/0289507 A1
(43) Pub. Date: Oct. 31, 2013

(54) Drainage Bag Garment Attachment and Process

(71) Applicant: Lloyd A. Carpenter, Mt. Vernon, OH (US)

(72) Inventor: Lloyd A. Carpenter, Mt. Vernon, OH (US)

(21) Appl. No.: 13/459,658

(22) Filed: Apr. 30, 2012

(51) Int. Cl. A61F 5/44

(52) U.S. Cl. CPC A61F 5/4408
USPC 604/345

(57) ABSTRACT

The current invention is a garment Drainage bag attachment and process that can be used with any garment or article of clothing. It can be attached to a user's own garment or it can be sold as part of the garment specially made to be sold to Drainage bag users. It consists of a strip of material with two buttons attached in parallel. These buttons are spaced so that they match the slots in the Drainage bag. The strip is attached to the garment a little below waist high on either the slight forward left or slight forward right of the bottom garment, such as pants, shorts or dress being worn by the user.

### BACKGROUND OF INVENTION

1. Field of the Invention

The present invention is directed to a Drainage Bag garment attachment in particular one that can be used with any garment.

2. Background

Having to use a Drainage bag is a life changing event such as with a nephrostomy and a nephrostomy tube. In the current art, it is held around the leg with hook and loop straps. As the bag fills up, the weight slips down and pulls on the straps causing the user pain. The Drainage bag has to be located below the drainage site so it is normally on the hip or thigh.

Drainage bag users can be very uncomfortable and self conscious as the Drainage bag in the current art is not designed for comfort or to be worn unnoticeable. There exists a need for Drainage bag users to be able to use their Drainage bag with any clothing or fashion with comfort.

There is still room for improvement in the art.

### SUMMARY OF THE INVENTION

The current invention is a garment Drainage bag attachment and process. It can be used with any garment or article of clothing. It can be attached to a user's own garment or it can be sold as part of the garment specially made to be sold to Drainage bag users.

The current invention is a strip of cloth with two buttons attached in parallel. These buttons are spaced so that they match the slots in the Drainage bag. These slots are, in prior art, used to attached the holding straps. The strip is attached to a piece of garment and the Drainage bag is attached to the strip by inserting the buttons into the slots of the Drainage bag. The strip is attached to the garment a little below waist high on either the slight forward left or slight forward right of the bottom garment, such as pants, shorts or dress being worn by the user.

This provides the user with a comfortable and inconspicuous way to wear their Drainage bag with any garment or type of garment.

### BRIEF DESCRIPTION OF THE DRAWINGS

Without restricting the full scope of this invention, the preferred form of this invention is illustrated in the following drawings:

FIG. 1 is a view of a standard Drainage bag and the prior art attachment means;

FIG. 2 displays the invention attached to a garment;

FIG. 3 displays a Drainage bag attached to the invention; and

FIG. 4 displays the garment where the Drainage bag is not visible.

### BRIEF DESCRIPTION OF THE PREFERRED EMBODIMENTS

There are a number of significant design features and improvements incorporated within the invention.

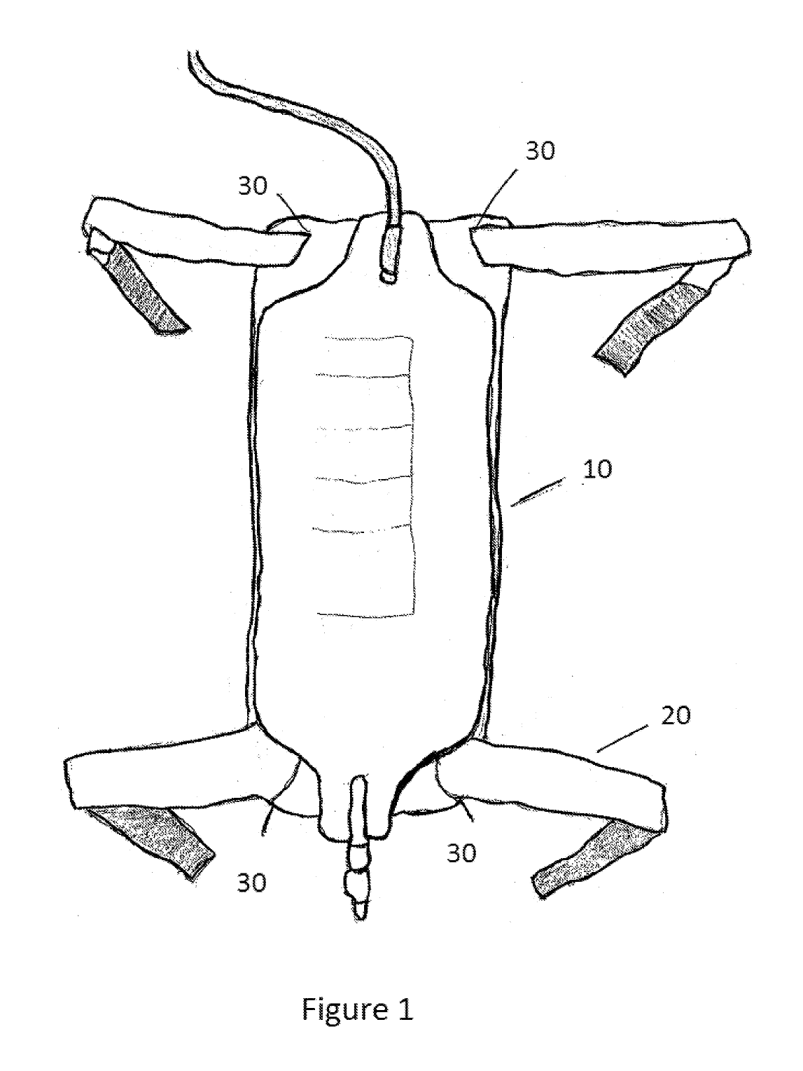

As shown in FIGS. 1, in the current art, a Drainage bag 10 is held around the leg or hip with hook and loop straps 20 that run through slots 30 in the bag 10. As the bag 10 fills up the weight slips down and pulls on the straps causing the user pain. The Drainage bag 10 has to be located below the drainage site so it is normally on the hip or thigh. A Drainage bag 10 is used to collect urine or other body waste or fluids through a tube, like a nephrostomy tube, connected to the body when a person cannot extract urine normally through the bladder and urethra. The Drainage bag 10 could be used for other wastes or liquids besides urine.

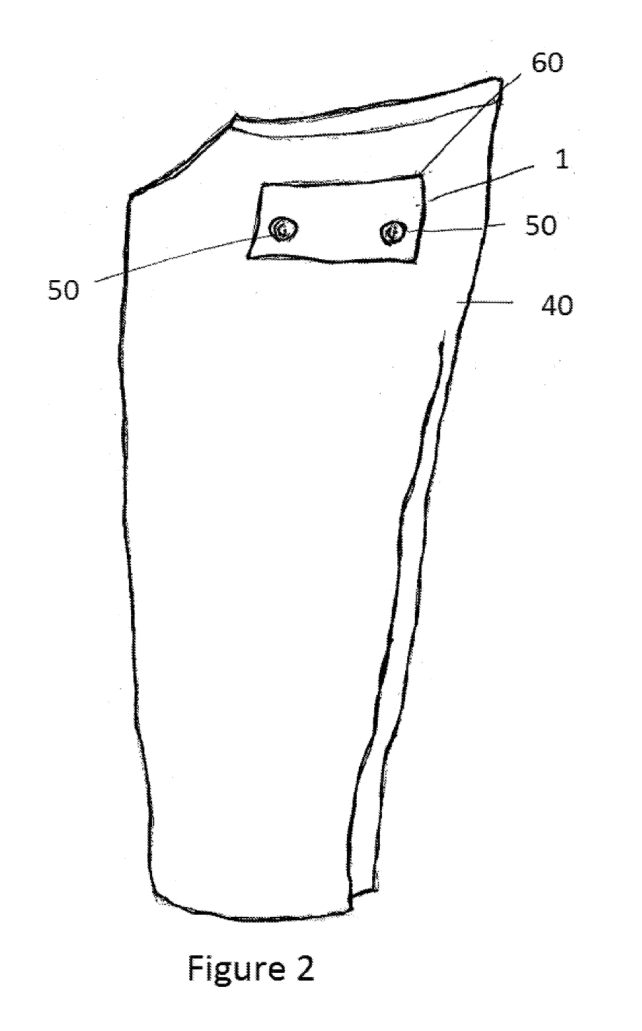

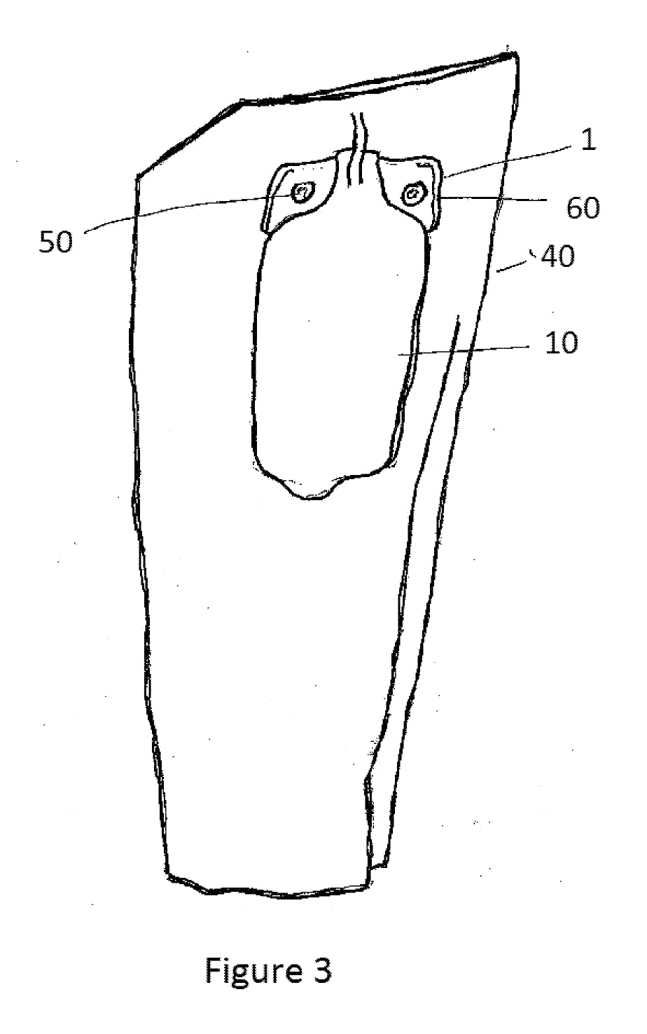

In the preferred embodiment, as shown in FIG. 2, the current invention 1 is a strip of material 60 on which two connection means such as buttons 50 are located. These connecting means 50 are in parallel with each other and are spaced apart so that they will fit into the slots 30 of the bag 10 to hold it in place as shown in FIG. 3. This eliminates the needs for the straps 20 and allows for the bag to hang more naturally and to work with the clothing/garment 40. Other connection means such as clips, hook and loops or snaps can be used.

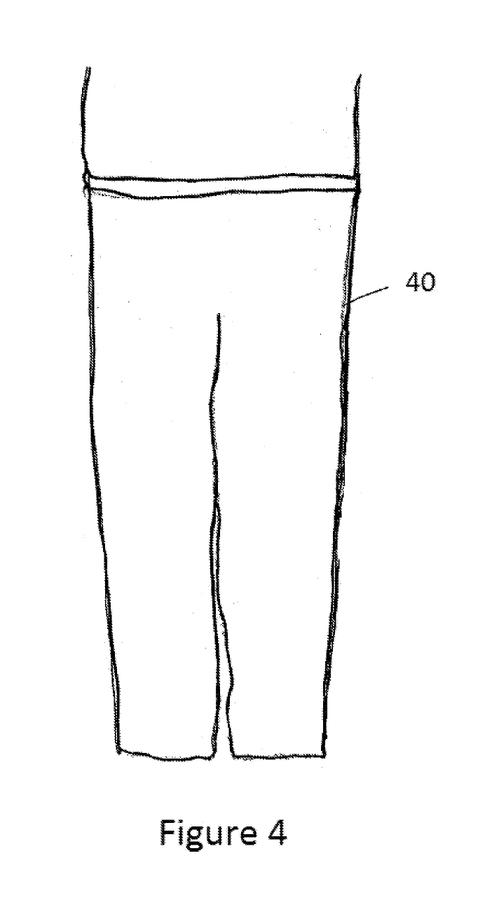

The strip of material 60 can be made of any sturdy preferable flexible material including cloth. The strip 60 can be attached to a user's existing clothing 40 or could come premade in clothing 40 made especially for Drainage bag users. As shown in FIG. 2, the strip 60 will be attached to the inside of the clothing/garment 40 using an attachment means such as sewing or gluing. In an alternate embodiment, the clothing could come premade with the connection means without the strip of material. The strip of material 30 would be connected to the inside of the garment 40 usually at the front side hip or side thigh level for a more natural fit and proper weight distribution. FIG. 4 shows the outside of the clothing/garment 40 with the Drainage bag being connected to the invention. The strip of material 60 is sewn in in the preferred embodiment but can be attached through gluing or similar means.

The connecting means 50 can be any connecting means that could hold the Drainage bag 10 using the slot 20. In the preferred embodiment it is the use of buttons but it could be snaps, hooks, bars or any similar means. The connecting means 50 would be strong enough to connect and hold the bag 10 without excess weight or showing through the garment 40.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A Drainage holding bag comprising:
A strip of material attached to a garment using an attachment means with a plurality of connection means to which a Drainage bag is attached.

2. A Drainage holding bag as in claim 1 further comprising:
Where said connection means are buttons.

3. A Drainage holding bag as in claim 1 further comprising:
Where said attachment means is sewing.

4. A Drainage holding bag as in claim 1 further comprising:
Where said attachment means is glue.

5. A process to attach Drainage holding bag at clothing comprising:
Having a strip of material, attaching it to a garment using an attachment means, having a plurality of connection means on said strip and connecting a Drainage bag to said strip using the connection means.

6. A process to attach Drainage holding bag at clothing as in claim 1 further comprising:
Where said connection means are buttons.

7. A process to attach Drainage holding bag at clothing as in claim 1 further comprising:
Where said attachment means is sewing.

8. A process to attach Drainage holding bag at clothing as in claim 1 further comprising:
Where said attachment means is glue.

9. A garment to hold Drainage holding bag comprising:
A strip of material attached to a garment using an attachment means with a plurality of connection means to which a Drainage bag is attached.

10. A garment to hold Drainage holding bag as in claim 9 further comprising:
Where said connection means are buttons.

11. A Drainage holding bag as in claim 9 further comprising:
Where said attachment means is sewing.

12. A Drainage holding bag as in claim 9 further comprising:
Where said attachment means is glue.
